(19) United States
(12) Plant Patent Application Publication
(10) Pub. No.: US 2002/0069443 P1
(43) Pub. Date: Jun. 6, 2002

(54) LILY PLANT NAMED 'SIMPLON'

(76) Inventor: CEES A. VAN DER VOORT, KATWIJK (NL)

Correspondence Address: FOLEY & LARDNER, WASHINGTON HARBOUR, 3000 K STREET, N.W., SUITE 500, WASHINGTON, DC, 20007-5109, US

(21) Appl. No.: 09/357,100

(22) Filed: Jul. 19, 1999

(51) Int. Cl. A01H 5/00

(52) U.S. Cl. PLT/315

(57) ABSTRACT

A new and distinct cultivar of Lily plant named ‘Simplon’ characterized by having large, upright flowers, white flower color, contrasting orange brown pollen, and vigorous growth habit.

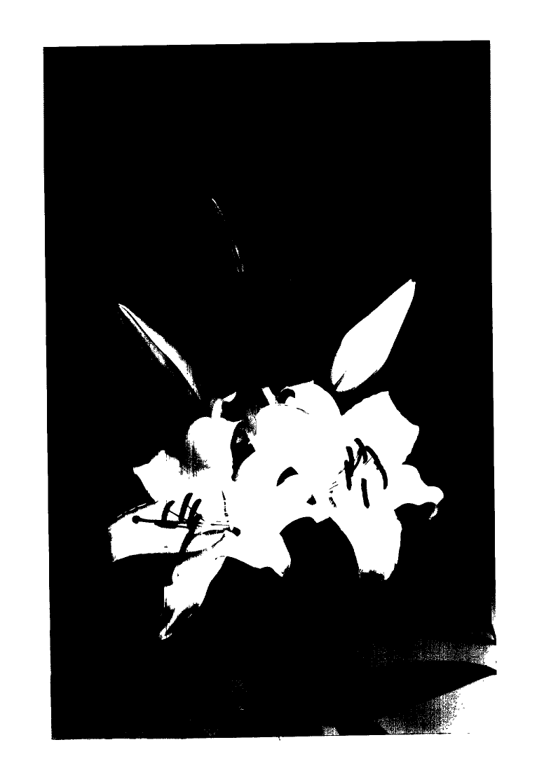

### BACKGROUND OF THE INVENTION

The present invention comprises a new and distinct cultivar of lily plant, botanically known as Lilium L., and hereinafter referred to by the cultivar name ‘Simplon’.

‘Simplon’ is a product of a planned breeding program which had the objective of creating new lily cultivars having large, white, upright flowers and vigorous growth.

‘Simplon’ was originated from a hybridization made by the inventor in a controlled breeding program in Rijnsburg, the Netherlands in 1984. The male and female parents were unnamed seedlings of Lilium L. oriental hybrids. Both parents are proprietary cultivars used in the breeding program.

‘Simplon’ was discovered and selected as one flowering plant within the progeny of a stated cross by the inventor, Cees A. van der Voort, on Jun. 7, 1984 in a controlled environment in Rijnsburg, the Netherlands.

The first act of asexual reproduction of ‘Simplon’ was accomplished when scales were taken from the initial selection on October, 1987 in a controlled environment in Rijnsburg, the Netherlands by, or under the supervision of, Cees A. van der Voort. Horticultural examination of selected units initiated May 1987 has demonstrated that the combination of characteristics as herein disclosed for ‘Simplon’ are firmly fixed and are retained through successive generations of asexual reproduction.

### BRIEF DESCRIPTION OF THE INVENTION

The following traits have been repeatedly observed and are determined to be basic characteristics of ‘Simplon’ which in combination distinguish this lily as a new and distinct cultivar:

1. large, upright flowers;

2. white flower color;

3. contrasting orange brown pollen; and

4. vigorous growth habit.

‘Simplon’ has not been observed under all possible environmental conditions. The phenotype of the new cultivar may vary significantly with variations in environment such as temperature, light intensity, and daylength without any change in genotype. The following observations, measurements, and comparisons describe plants grown in the experimental research station CPRO-DLO, Wageningen, the Netherlands under conditions which approximate those generally used in commercial practice.

Of the many commercial cultivars known to the present inventor, no other cultivars are similar in comparison to ‘Simplon’.

### BRIEF DESCRIPTION OF THE DRAWING

The accompanying color photographic drawing shows typical plant, bulb and flower characteristics of ‘Simplon’, with colors being as true as possible with illustrations of this type.

### DETAILED BOTANICAL DESCRIPTION

In the following description, color references are made to The Royal Horticultural Society Colour Chart (RHS). The color values were determined between 12 am and 2 pm at the Dutch experimental research station CPRO-DLO, Wageningen, the Netherlands.

Origin: Seedling from breeding program

Parentage:

Female parent.—Unnamed seedling

Male parent.—Unnamed seedling.

Classification:

Botanical.—A hybrid of the genus Lilium L.

Commercial: Oriental Lily cv. ‘Simplon’.

Plant:

Form.—A single stem carrying numerous, alternately arranged pedicels.

Height.—Medium to high for the mature plant, approximately 90 cm, depending on cultivation conditions.

Growth Habit.—Vigorous.

Stem.—Circular in transverse section; anthocyanin pigmentation absent; internodal length uniform.

Foliage.—Quantity: Approximately 70, depending on cultivation conditions Size of leaf: Mature leaves are 10 cm long and 3.5 cm wide, depending on cultivation conditions Shape of leaf: Generally ovate, hardly any pubescence Color: Light green.

Bulb.—Size: 16 to 18 cm in circumference Color: White

Bud:

Form.—Tapering like a lancet Size: Large, 14 cm in length, depending on cultivation conditions Opening: Unfolds normally Color: White, RHS 155D, just before opening Tepals: Folded, three visible

Flower:

Blooming habit.—Raceme.

Size: Medium to wide, 21 cm in diameter, depending on cultivation conditions.

Borne.—Upright to horizontal along a longitudinal axis.

Shape.—Generally, hexagonal star in form and bowl-shaped in cross-section.

Tepalage.—Number of Tepals: 6 Arrangement: Three inner and three outer Color: Upper Surface: White, RHS 155D; color of mid-vein is green Lower Surface: White, RHS 155D Spotting or Marking: None

Pedicel.—Length: Medium to long Color: Form: Tapering like a lancet.

Texture.—Papillose.

Disease Resistance.—Medium.

Fragrance.—Absent.

Lasting Quality.—Excellent.

Reproductive Organs:

Stamens and anthers.—Arrangements: Anthers form a generally circular pattern at their upper ends Number: 6 Length: 8 cm, depending on cultivation conditions Filaments Color: White.

Pollen color.—Reddish-brown, RHS 171B

Anther color.—Reddish-brown.

Pistils.—Green.

Stigma.—Purple.

Ovaries.—Triangular in shape.

I claim:

1. A new and distinct cultivar of Lily plant named ‘Simplon’, as illustrated and herein.
